(19) United States
(12) Patent Application Publication
(10) Pub. No.: US 2014/0116452 A1
(43) Pub. Date: May 1, 2014

(54) MOTION RESTRICTION KIT

(71) Applicant: OSSUR HF, Reykjavik (IS)

(72) Inventors: Arni Thor INGIMUNDARSON, Gardabaer (IS); Valgeir PETURSSON, Reykjavik (IS); Helga Run PALSDOTTIR, Reykjavik (IS); Bjorn OMARSSON, Reykjavik (IS)

(73) Assignee: OSSUR HF, Reykjavik (IS)

(21) Appl. No.: 14/061,134

(22) Filed: Oct. 23, 2013

Related U.S. Application Data
(60) Provisional application No. 61/719,004, filed on Oct. 26, 2012.

(51) Int. Cl. A61F 5/05

(52) U.S. Cl. CPC A61F 5/05
USPC 128/882

(57) ABSTRACT

A movement restriction kit is provided for lower legs and lower extremities. The embodiments are provided for limiting or immobilizing hip rotation, abduction and adduction post hip arthroscopy. The kit includes first and second slippers spaceable from one another by a plurality of different spacers and arranged to secure to one another or the spacer by a tab carrying a plurality of laces extending from each of the first and second slippers.

### FIELD OF THE DISCLOSURE

The disclosure relates to a movement restriction kit, and more specifically to a kit for immobilizing legs and lower extremities after surgery and during rehabilitation.

### BACKGROUND

A number of surgical options is available for individuals with different hip joint issues. Some of the options include arthroscopic surgery techniques such as labral tear resection, labral repair, capsular modification, osteoplasty, and microfracture procedures. Postoperative rehabilitation following arthroscopic procedures of the hip joint concerns the range of motion, weight bearing precautions, and initiation of strength activities.

While range of motion activities begin soon after surgery, a brace may be used to protect the hip joint and limit the range of motion, particularly hip abduction, adduction and rotation. Patients may be provided with a motion restriction kit that may be used particularly at night, in order to prevent the hips from external rotation during sleep and places the anterior capsuloligamentous structures under increased tension. It is often desirable to avoid prolonged periods of increased tension for individuals undergoing capsular modification procedures.

Patients typically have discomfort and limited range of motion following surgery, and require simple and flexible means for donning and adjusting parts of the kit. Moreover, some patients may be geriatric and have limited strength and flexibility, thereby requiring a flexible kit that can accommodate a variety of patients without discomfort. Since a motion restriction kit is often worn at nighttime, it must be easily adjustable to permit the patient to doff quickly at least parts of the kit in order to use the restroom or attend to other needs.

Although there are known motion restriction kits, these kits are often found difficult to secure and hard to adjust, particularly for patients under pain, discomfort and of limited strength following surgery. Accordingly, it is desirable to provide a motion restriction kit that securely immobilizes legs and lower extremities while being easy-to-use and readily adjustable, and providing comfort.

### SUMMARY

In accordance with various embodiments described herein, a movement restriction kit is provided for lower legs and lower extremities. The embodiments are provided for limiting or immobilizing hip rotation, abduction and adduction post hip arthroscopy.

According to an embodiment, the motion restriction kit includes at least one slipper having a plurality of laces carried by a tab securable over the surface of the at least one slipper, and an ankle strap adapted to wrap about an ankle and secure to the at least one slipper. In a preferred embodiment, the at least one slipper includes first and second slippers mirror opposite to one another. The tab of each of the first and second slippers is arranged to removably secure to a respective opposite one of the first and second slippers.

The first and second slippers may has open posterior and anterior portions, and the plurality of laces may be adapted to extend between lateral and medial sides of the foot. The first and second slippers may also each include a sleeve adapted to cover a dorsum of a foot.

The motion restriction kit may include a connector securable along medial sides of the first and second slippers. The connector has a central pad extendable between the first and second slippers and arranged to space the first and second slippers apart. The motion restriction kit may also have at least one support strap arranged to secure about a lower portion of the at least one slipper.

The motion restriction kit may have a knee wrap including a main body and a pair of loops extending from the main body for encircling legs. The main body may be generally stiffer than the loops. The loops are defined by a first segment depending from the main body and adjustably securable by strap tabs, and a second segment depending from the main body to thereby form a loop with the main body. The main body may extend downwardly from the pair of loops to provide padding between the legs.

The motion restriction kit may have a cylindrical pad, and the tab of the at least one slipper is arranged to removably secure over the surface of the cylindrical pad. The cylindrical pad may define upper and lower planar surfaces, and the ankle strap extends over one of the upper and lower surfaces. The cylindrical pad may have greater width than the main body of the knee wrap.

A method for donning the motion restriction kit may include placing a first slipper having a plurality of laces over a first foot, the plurality of laces carried by a tab securable over the surface of the slipper, tensioning the laces by pulling the tab, and attaching the tab to a substrate to a surface proximate to the first slipper.

The method may include the step of placing a second slipper over a second foot, and the step of securing the tab of the first slipper to a surface of the second slipper such that the surface of the second slipper forms the substrate.

In a variation, the method may include the step of placing a cylindrical pad between the first and second slippers whereby a surface of the cylindrical pad forms the substrate upon which the tab secures. Both the first and second slippers may secure to the cylindrical pad at a plurality of orientations relative to an upper surface of the cylindrical pad.

The method may further include the step of wrapping an ankle strap about a rear portion of the first slipper to prevent travel of the foot rearward of the slipper. The ankle strap may secure to both the first and second slippers whereby the first and second slippers have open rear ends.

The method may also involve securing a knee wrap to a pair of legs whereby the knee wrap includes a main body placed between the pair of legs, and a pair of loops each respectively secured about one of the legs.

Various other embodiments of a motion restriction kit are described herein, and the kit may have any number of the various components described herein and is not limited to any single disclosed combination.

### BRIEF DESCRIPTION OF THE DRAWINGS

These and other features, aspects, and advantages of the present disclosure will become better understood with regard to the following description, appended claims, and accompanying drawings.

FIG. 1 is a plan view of an embodiment of the motion restriction kit according to the disclosure.

FIG. 2 is a perspective view of an embodiment of a knee wrap according to the kit of FIG. 1.

FIG. 3 is a detailed plan view of the slipper and slipper connector of FIG. 2 with another slipper attached thereto.

FIG. 4 is a schematic view of securing the slippers of FIG. 3 to one another.

FIG. 5 is a schematic view showing the application of an ankle strap to the slippers of FIG. 4.

FIG. 6 is a schematic view of the knee wrap of FIG. 1 secured onto legs of a patient.

FIG. 7 is a schematic view of the kit on the legs and lower extremities of a patient.

FIG. 8 is a schematic view showing an anterior side of another embodiment of a knee wrap.

FIG. 9 is a schematic view showing a posterior side of the knee wrap embodiment of FIG. 8.

FIG. 10 is a bottom plan view showing attachment of the slippers of FIG. 1 onto a perineal pad.

FIG. 11 is a top plan view showing the slippers and perineal pad of FIG. 10 in a neutral position.

FIG. 12 is an elevational view showing the slipper and the perineal pad of FIG. 11 and an ankle strap applied thereto.

FIG. 13 are a schematic view showing the slipper and the perineal pad m an abduction position.

FIG. 14 is a perspective view of a foot wrap embodiment for use with the leg strap of FIG. 8.

FIG. 16 is a perspective view of yet another foot wrap embodiment.

FIG. 16 is a schematic view showing a posterior side of the foot wrap embodiment of FIG. 15 connected to the leg strap of FIG. 9 and attached to a perineal pad.

FIG. 17 is a perspective view of the kit according to FIG. 1 including lace strap extensions.

FIG. 18 is a perspective view of the lace strap extensions wrapped about ankles of a patient.

FIG. 19 is a perspective view of the lace strap extensions wrapped about a perineal pad.

FIG. 20 is a perspective view of a variation of the ankle strap on the perineal pad.

It should be noted that the drawing figures are not necessarily drawn to scale, but instead are drawn to provide a better understanding of the components thereof, and are not intended to be limiting in scope, but rather to provide exemplary illustrations. It should further be noted that the figures illustrate exemplary embodiments, and in no way limit the structures or configurations of according to the present disclosure.

### DETAILED DESCRIPTION OF VARIOUS EMBODIMENTS

A. Overview

A better understanding of different embodiments of the disclosure may be had from the following description read in conjunction with the accompanying drawings in which like reference characters refer to like elements.

While the disclosure is susceptible to various modifications and alternative constructions, certain illustrative embodiments are shown in the drawings and are described below in detail. It should be understood, however, that there is no intention to limit the disclosure to the specific embodiments disclosed, but on the contrary, the intention is to cover all modifications, alternative constructions, combinations, and equivalents falling within the spirit and scope of the disclosure.

It will be understood that, unless a term is expressly defined in this disclosure to possess a described meaning, there is no intent to limit the meaning of such term, either expressly or indirectly, beyond its plain or ordinary meaning.

The kit embodiments described herein are configured for use as a motion restriction kit for the legs and lower extremities. It should be kept in mind, however, that the same concepts and methods described herein may be similarly used for other orthopedic devices and are not limited solely to the anatomical locations discussed herein.

General anatomical terms for the human body may be used herein for describing particular locations of the elements of the kit in comparison to the human body.

Each of these terms is used in reference to a human leg, knee, foot or ankle, by way of example, which is divided in similar sections with a proximal-distal plane generally extending along the meniscus of the knee between the femur and tibia. The terms “proximal” and “distal” generally refer to locations that correspond to the location of leg relative to the point of attachment of the leg to the body. The terms “upper” and “lower” may be used in combination with “proximal” and “distal” to connote gradations in location of “proximal” and “distal.”

The elements of the kit can also be considered to fall within “anterior” and “posterior” sections by an anterior-posterior plane. The anterior-posterior plane generally corresponds to the coronal or frontal plane of a human leg, which lies along the central longitudinal axis of a body. A posterior side or element is therefore located behind this anterior-posterior plane, whereas an anterior side or element is located in front of the anterior-posterior plane.

The terms “inwardly” or “inner” may be used herein to distinguish certain elements of the kit that may be directed to the posterior side of the patient. Contrariwise, the term “outwardly” or “outer” are used to denote the side of certain elements of the kit that are opposite to the inwardly side.

The terms “medial” and “lateral” are relative terms that are generally understood as indicating location near the midsaggital plane or midline. Therefore, elements that are located near the midline are referred to as “medial” and those elements that are further from the midline are considered to be “lateral.” The term “central” is used to denote the area along the midline of a joint thereby dividing and sharing regions of the medial and lateral regions.

B. Embodiments of the Motion Restriction Kit

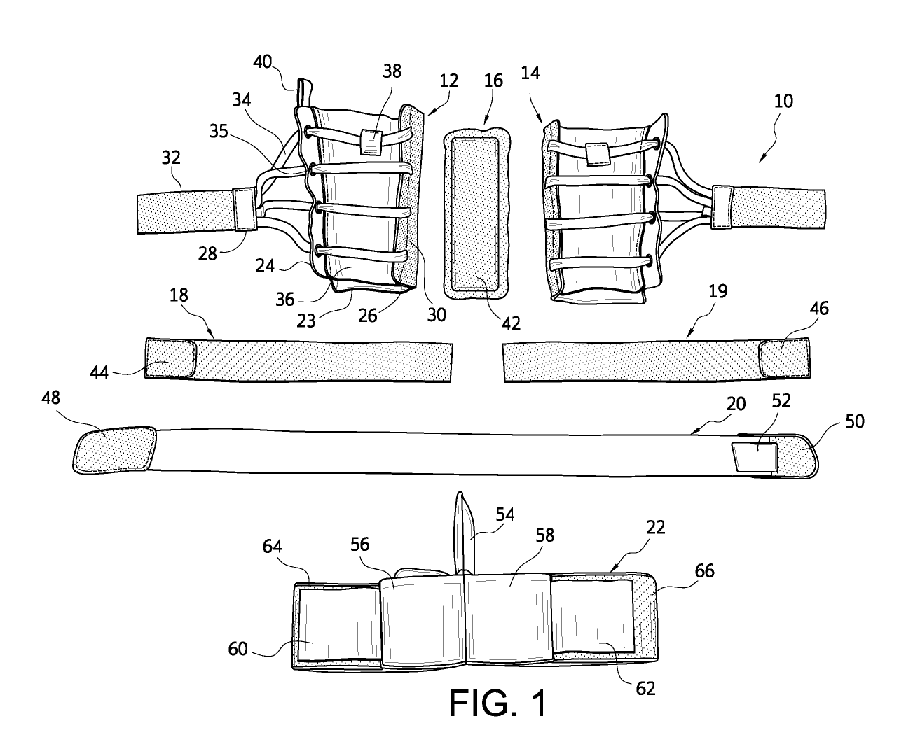

In the embodiment depicted in FIG. 1, a motion restriction kit 10 is arranged for immobilizing legs and lower extremities after surgery. As for positioning the lower extremities, inclusive of the feet and ankles of the patient, the kit 10 includes first and second slippers 12, 14 arranged to secure to the dorsum and plenum of the midfoot (including the instep) and the forefoot. While the slippers are not so limited, it is preferable that the slippers do not extend about the heel of a patient. A connector 16 is adapted to be secured between the first and second slippers. At least one support strap 18, 19 may be secured to the first and second slippers 12, 14, and preferably extend over portions of the dorsal aspect of the slippers. An ankle strap 20 is adapted to wrap about an ankle of the patient and secure to the slippers. The kit 10 further includes a knee or leg wrap 22 that is adapted to secure about the legs of the patient and retain the legs closely together at a location at or near the patient's knees.

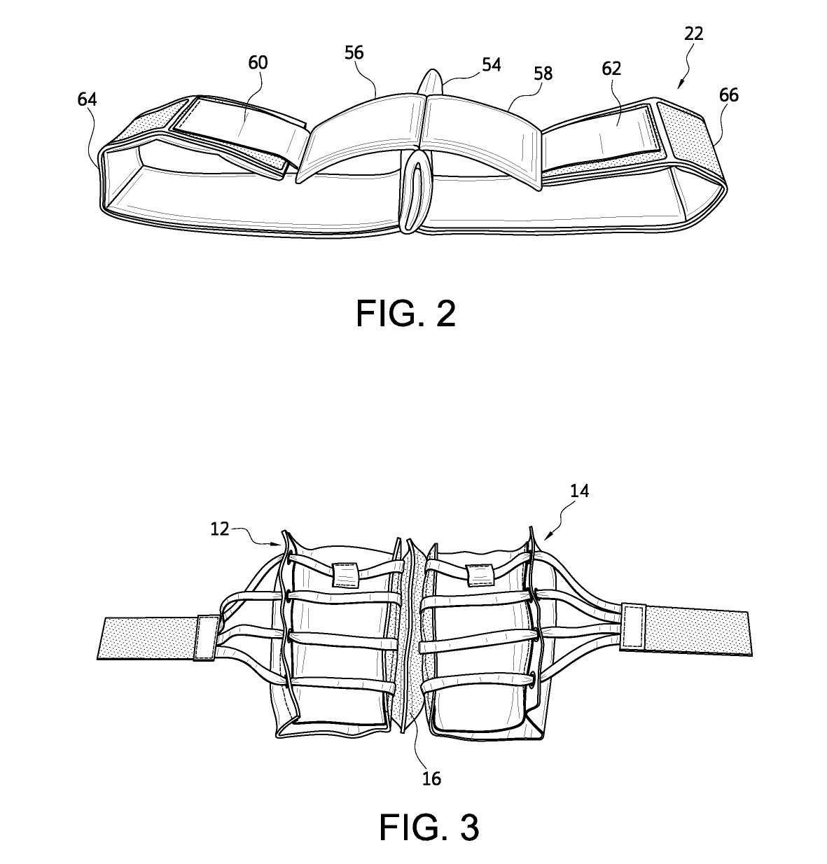

In referring to the slippers according to FIGS. 1 and 2, each slipper 12, 14 are a mirror image of the other slipper. The slipper 12 includes first and second sides 24, 26 formed by a main body panel 23 that continuously extend underneath the planum of the foot from lateral and medial sides of the foot. A strap 28 extends from the main body panel 23 and carries a tab 32 having a plurality of hook elements or other suitable fastener element.

The strap 28 includes a plurality of laces 34 that are anchored to the second side 26 of the slipper 12, and extend through a plurality of lace guides 35 or grommets located on the first side 24. The slipper 12 has a foot wrap or sleeve 36 that is arranged to cover the dorsum of the foot and includes at least one guide 38 through which one of the laces 34 extends. It follows by known methods that the second side is drawn toward the first side as the strap 28 is pulled. The outside surface of the second side 26 has a hook element 30 that is adapted to engage a surface of the slipper connector 16.

A pull-tab 40 extends at least from an edge of the first side 24 of the slipper so as to allow the slipper 12 to be pulled onto the foot of a patient. Additional pull-tabs 40 may be provided, such as along the second side 26 of the slipper to allow for additional means for sliding the slipper snugly onto the foot of the patient.

The slipper may be constructed singularly or from a combination of a variety of materials such as textiles, foam, foam fabric including spandex, Lycra, nylon, polyester, OUTLAST, COOLMAX, AEROSPACER, microfiber, three-dimensional fabrics, and other suitable fabrics. According to a preferred embodiment, the slippers are formed from breathable foam laminated with a knitted loop material. The slippers may include localized regions having thermoformed foam that provide a contour to the main body panel that anatomically conforms to the foot to the foot. The thermoformed foam regions may be from the main body panel itself, or provided in combination and secured to the main body panel.

The foot wrap 36 is preferably constructed from an elasticized textile in order to adjustably fit and extend at least over the dorsum and thereby comfortably and securely protect and cover the foot.

It is advantageous from the construction of the slippers, such as extending along the midfoot and the forefoot, and the foot wrap which snugly fits onto the foot, that the slippers are highly breathable in contrast to slippers or boots which extend fully around both the entirety of the foot and lower leg. Moreover, in view of the lacing system provided on the slippers, the sides of the slippers can be fully tensioned by generally evenly applying pressure along the entirety or substantial entirety of the sides of the slippers over the foot. This is particularly advantageous for weak or impaired patients that lack the strength or dexterity and require easy adjustment of the slippers. The lacing system further simplifies donning and doffing the slippers since a single strap tab is required and such strap tab can be placed over a variety of locations for securing the strap onto another slipper or object, as will be discussed below. This is beneficial since there is not a fixed location upon which the strap tab much be placed, and further allows for quick removal of the strap tab in the event that the patient must quickly doff the motion restriction kit.

The connector 16 preferably has a central padded region 42. The central padded region may be particularly defined by a compressible foam pad covered by hook receivable material so that the connector 16 can engage with the hook elements 30 located along the slippers 12, 14. In a variation, the padded region may be thermoformed so as to have differing areas of compression and contours to accommodate anatomy of the wearer's lower extremities.

The connector is advantageous in that it not only serves to couple the slippers to one another, but also provides additional padding at localized areas of the feet (as opposed to surrounding the entirety of the foot or slippers) and allows for better breathability. Also, the localized placement of the padding also reduces the weight and size of the kit, again rather than extending about the entirety of the lower extremities, and allows for a lower profile which is helpful when the patient is sleeping.

The support straps 18, 19 are adapted for at least extending along the plantum or bottom surface of the slippers. The straps 18, 19 may also be used to secure the slippers 12, 14 at other locations or can be used as extensions to any of the straps described herein. Preferable, each of the support straps 18, 19 has at least one surface that is hook receivable, and may also have an opposed surface to hook receivable surface defined with at least portions having hook elements. Alternatively or in combination therewith, the support straps may have a hook element 44, 46 located at an end portion thereof.

The ankle strap 20 has opposed ends with clip tabs 48, 50 that can be removed for proper sizing of the ankle strap. The clip tabs 48, 50 include an anchor strap 52 that permits the removal of the clip tabs and allows for replacement of the clip tabs on the resized ankle strap.

The knee wrap 22 according to the embodiment of FIGS. 1 and 2 includes a main body 54 upon which loops 58 extend for encircling the legs with the main body 54. Specifically, the main body 54 is padded and may be generally stiffer than the loops 58 in order to provide cushioning between the knees and/or legs. Preferably the main body 54 extends both above and below the loops.

A first segment 56 defines the loops 58 depending from the main body 54 and adjustably securable by strap tabs 60, 62 to a second segment 64, 66 also depending from the main body 54 to thereby form a loop with the main body 54. The first segment 56, 58 is shorter than the second segment 64, 66 and is adapted to extend about the anterior side of the patient's legs. This allows the patient to adjust the strap tabs 60, 62 from the front of the legs rather than from the posterior side of the legs. The second segments therefore wrap from the main body which is centrally locatable between the patients legs, across the posterior side of the legs and at least along portions of the lateral and medial sides of the patient's legs.

The knee wrap may be constructed from a variety of materials similarly in range to those useable for forming the slippers.

In reference to FIGS. 3-5, the kit is positioned to place the feet, ankles and knees together of the patient. According to FIG. 3, the slippers 12, 14 are attached to one another by applying the connector 16, extending the laces if needed for larger feet, and binding the slippers together by securing against the connector 16. The patient then slides the feet together into the slippers by pulling the finger strap. Next, if necessary, the support strap is placed at the bottom of the slippers for additional restraint of the slippers relative to one another.

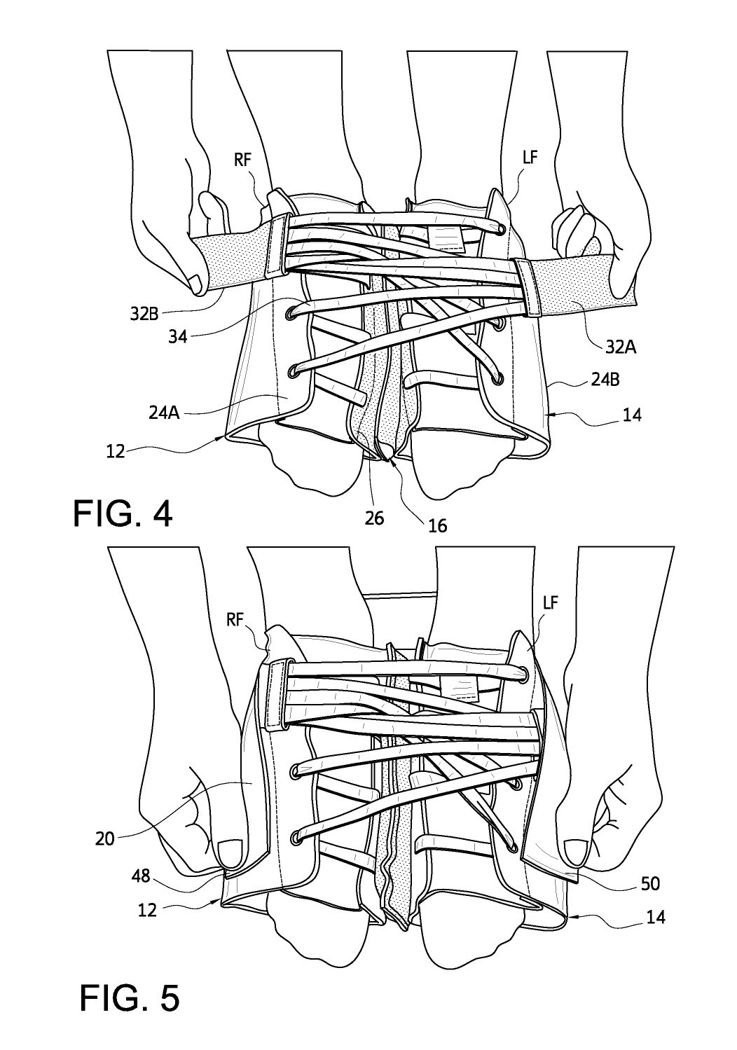

Turning to FIG. 4, the tabs 32A, 32B and the accompanying laces are crossed over each of the right and left feet (RF, LF), and the laces are tensioned until the slippers are securely comfortable on the feet. Next, the tabs 32A, 32B are secured to the opposite slipper.

Next, in reference to FIG. 5, the ankle strap 20 is wrapped around the heels of the patient so as to comfortably hug the foot, and the tabs 48, 50 are secured to the hook receivable surface of the slippers. The tabs 48, 50 are preferably located close to the small toes of the wearer on the corresponding slippers.

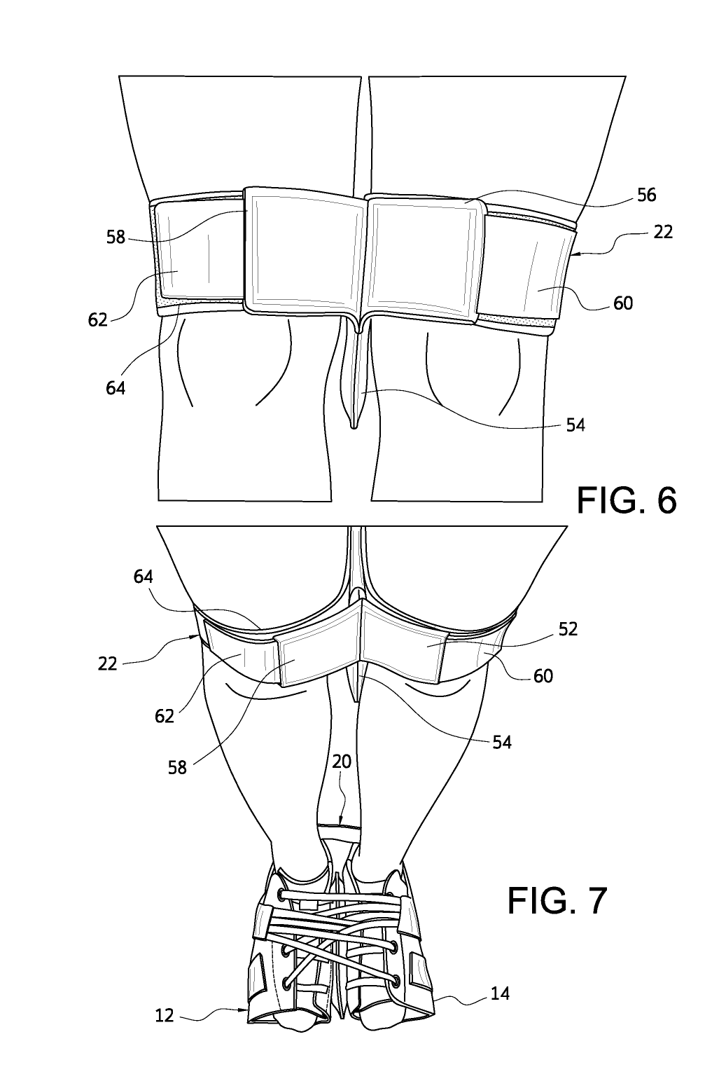

As illustrated in FIG. 6, the knee wrap 22 preferably is applied to the legs above the knees so that the main body 54 may be located at the knees to provide cushioning therebetween.

When assembled, the kit is applied to both the legs and the feet, as shown in FIG. 7, thereby restricting movement by limiting hip rotation, abduction and adduction of the hip.

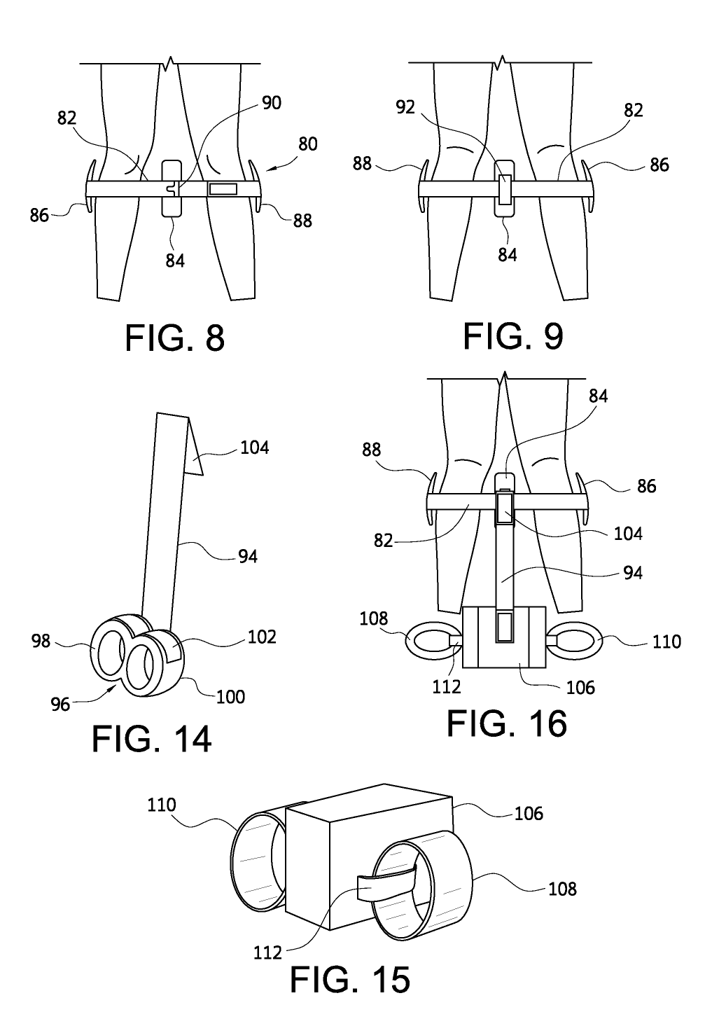

According to the embodiment of FIGS. 8 and 9, an alternative knee wrap 80 is provided. The knee wrap 80 includes a central circumferential strap 82 upon which a central pad 84 is slid ably mounted between the medial sides of the legs and particularly at the knees. The first and second leg supports 86, 88 are slidably mounted on the strap 82 (by loops through which the strap extends), and are preferably located on the lateral sides of each of the legs. A tensioning element 90 is provided on the anterior side to allow for tensioning of the strap 82 so as to draw the legs toward one another, and a loop 92 is provided on the posterior side of the pad 84 to retain the strap.

The tensioning element 90 may be a friction/lip buckle that has a quick release mechanism to allow for easy adjustment of strap tightness by using only one pull to tighten the strap. An end of the strap may be attached to the central pad, and a loose end may extend beyond the buckle and attach by hook and loop fasteners to the strap.

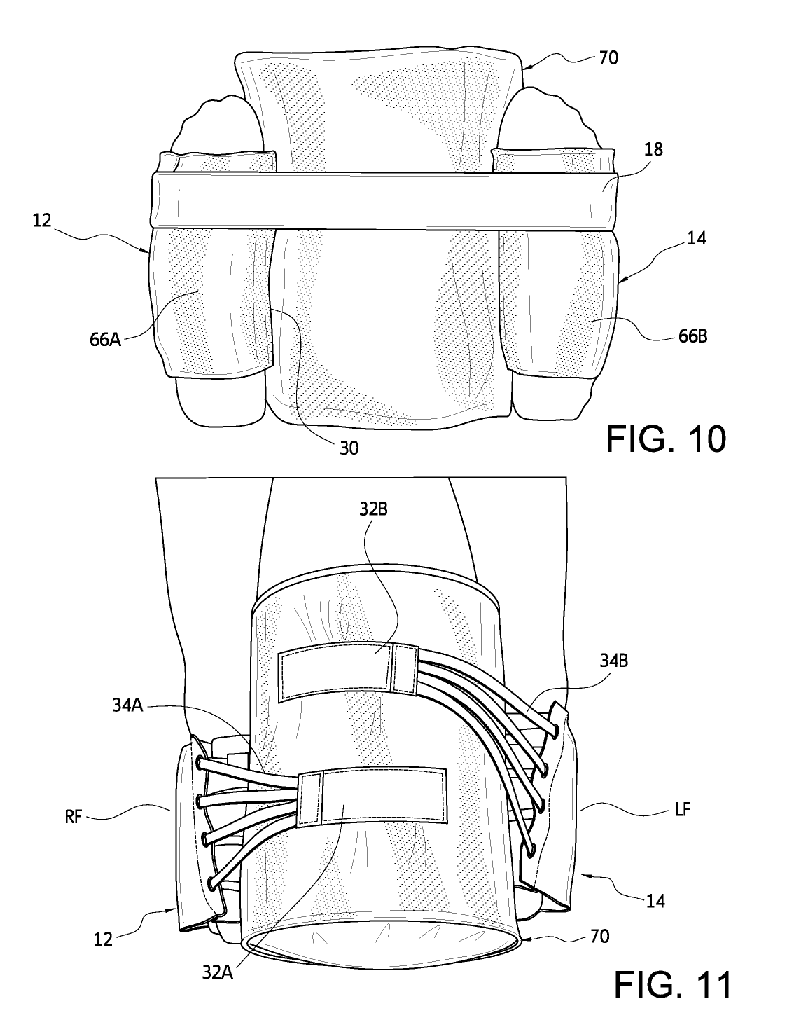

In reference to FIG. 10, the kit may be arranged to keep the hip joint in a neutral position with a perineal pad 70. Specifically, the slippers 12, 14 are attached to a post perineal pad 70 on the medial side of the slippers by the hook element 30 to a hook receivable surface of the perineal pad 70. A support strap 18 is placed under both the slippers 12, 14 along the platum surface 66 and a bottom surface of the pad 70. It is preferable that the support strap 18 extends to or near the lateral side of both slippers.

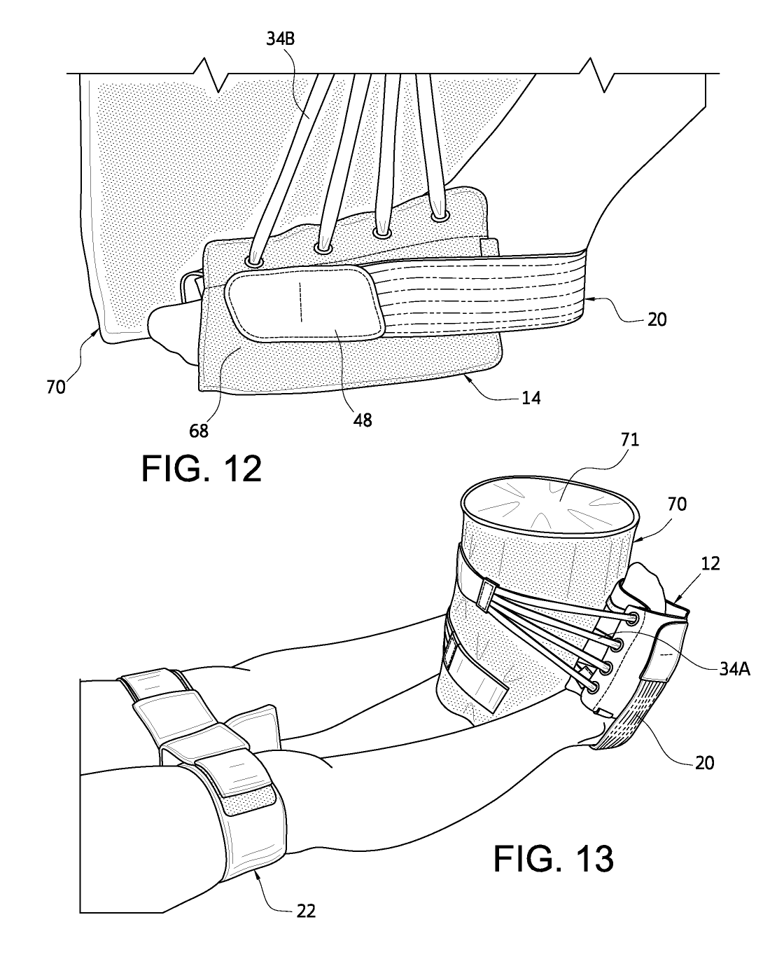

FIG. 11 shows that the strap tabs 32 and corresponding laces 34 are drawn over a top surface of the perineal pad 70 such that the slippers are attached to both the top and bottom surfaces of the perineal pad. FIG. 12 depict the ankle strap 20 as being applied about the patient's heels and an end of the perineal pad with the clips 48 being attached to the lateral sides 68 of the slippers 12, 14.

FIG. 13 shows an optional arrangement wherein the slippers 12, 14 are arranged relative to the perineal pad to allow for some abduction. Specifically, the knee wrap 22 is preferably placed right above the knee, and the slippers 12 are arranged at a skewed angle relative to at least the top surface 71 of the perineal pad 70.

FIG. 14 shows an alternative embodiment to the slippers wherein a foot wraps 96 is employed having foot loops 98, 100 connected to one another. The foot loops 98, 100 are stitched or otherwise secured to one another. At least one straps 102 is provided on the foot loops 98, 100 and arranged to circumferentially tighten the foot wraps 96 on the feet of the patient, with one end of the straps 102 being secured at the junction of the foot loops 98, 100. The foot wrap 96 is preferably constructed from foam with overlocked edges. A hook receivable textile is placed over the foot loops 98, 100 so the straps 102 can be adjustably secured thereto.

When worn, the clinician slips the patient's feet into the foot wraps without the patient having to bend down, close and perform other donning and doffing adjustments. Because the foot wraps are secured to one another, additional straps are not necessary to retain the patient's feet together.

An elongate draw strap 94 may be secured to the foot wraps and have an end 104 that secures to the knee wrap. The patient can pull the draw strap in order to slip the foot wrap onto the feet, thereby removing the need to bend over, and then attach the strap end 104 to the knee wrap in order to prevent the foot wrap from sliding off from the feet when the patient sleeps.

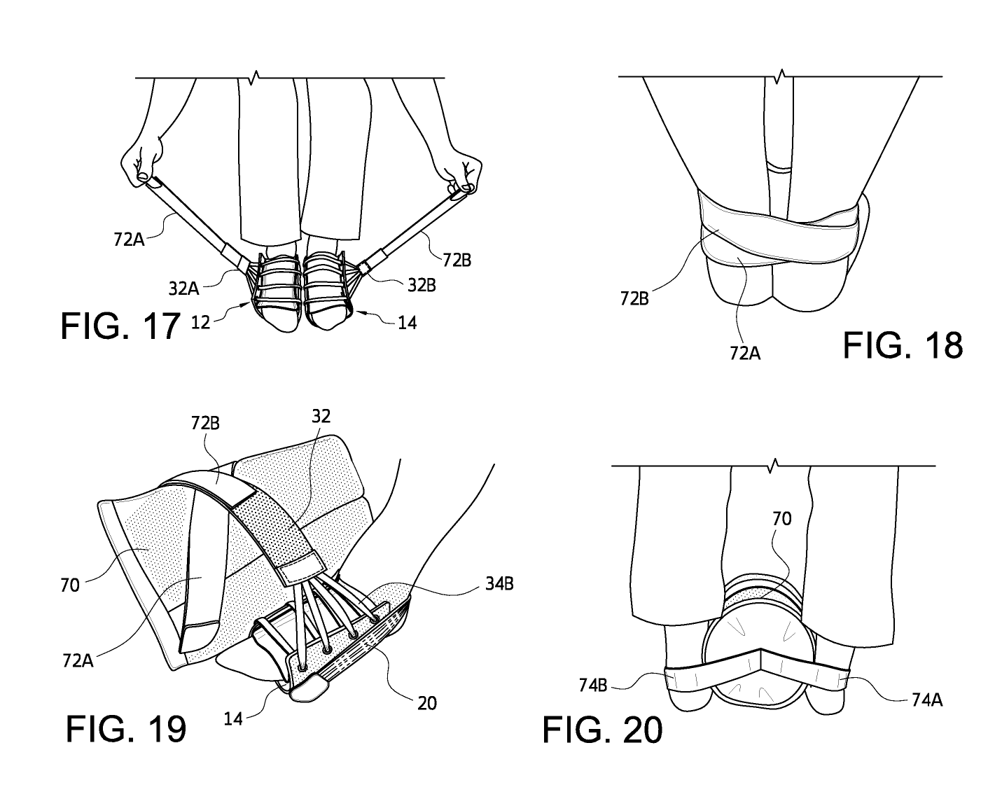

FIGS. 16 and 17 show a variation of the foot wrap according to FIG. 14 wherein the foot loops 108, 110 are detachable from another and securable to a wedge 106 via loops 112 located on the wedge 106. The draw strap 94 is securable to the leg wrap, such as the leg wrap in accordance with FIGS. 8, 9, at preferably the posterior side by a hook and loop engagement at the central pad 84 and the strap end 104.

From this embodiment, the foot wrap can be arranged as a separate unit for each foot, and the loops may be adjustably secured to the wedge or a perineal pad. Suitable straps and fasteners, if desired, can attach the wraps, to one another. The wedge can be used between the feet so as to externally rotate the hip and avoid adduction. The height and angle of the wedge can vary to accommodate different angles or rotation. Moreover, the wedge can be reversed in orientation according to the desire of establishing or reducing adduction.

FIG. 17 depicts the attachment of strap extensions 72 that may be added to the strap tabs 32. The strap extensions 72 may be used to wrap about the heel in supplement to or in replacement of the ankle strap. Alternatively, the strap extensions may be used to extend more significantly over a wedge or perineal pad, as shown in FIG. 19, to better secure the slippers to the wedge or perineal pad.

FIG. 20 shows any alternative embodiment wherein the ankle strap 74 comprises segments A, B which are secured to the perineal pad itself, thereby removing the need for a separate ankle straps.

Due to the adjustability of the embodiments described herein, the elements of the kit may be universally sized. As noted, despite the universality, the elements may trim accordingly to accommodate a particular patient's anatomy. Moreover, in view of the hook and look fasteners used for securing many of the elements relative to one another, a variety of attachment locations are provided to attach the elements to one another to optimize patient comfort.

1. A motion restriction kit comprising:
at least one slipper having a plurality of laces carried by a tab securable over the surface of the at least one slipper;
an ankle strap adapted to wrap about an ankle and secure to the at least one slipper.

2. The motion restriction kit of claim 1, wherein the at least one slipper includes first and second slippers mirror opposite to one another.

3. The motion restriction kit of claim 3, further comprising a connector securable along medial sides of the first and second slippers, the connector including a central pad extendable between the first and second slippers and arranged to space the first and second slippers apart.

4. The motion restriction kit of claim 2, wherein the tab of each of the first and second slippers is arranged to removably secure to a respective opposite one of the first and second slippers.

5. The motion restriction kit of claim 4, wherein the first and second slippers each define a hook receivable outer surface.

6. The motion restriction kit of claim 1, further comprising at least one support strap arranged to secure about a lower portion of the at least one slipper.

7. The motion restriction kit of claim 1, wherein the at least one slipper has open posterior and anterior portions, the plurality of laces adapted to extend between lateral and medial sides of the foot.

8. The motion restriction kit of claim 1, wherein the at least one slipper includes a sleeve adapted to cover a dorsum of a foot.

9. The motion restriction kit of claim 1, further comprising a knee wrap including a main body and a pair of loops extending from the main body for encircling legs, the main body being generally stiffer than the loops.

10. The motion restriction kit of claim 9, wherein the loops are defined by a first segment depending from the main body and adjustably securable by strap tabs to a second segment of the loops depending from the main body to thereby form a loop with the main body.

11. The motion restriction kit of claim 9, wherein the main body extends downwardly from the pair of loops.

12. The motion restriction kit of claim 1, further comprising a cylindrical pad, the tab of the at least one slipper arranged to removably secure over the surface of the cylindrical pad.

13. The motion restriction kit of claim 12, wherein the cylindrical pad defines upper and lower planar surfaces, the ankle strap extending over one of the upper and lower surfaces.

14. The motion restriction kit of claim 1, further comprising a knee wrap having a main body and a pair of loops extending from the main body for encircling legs, the main body arranged for spacing apart legs, and a cylindrical pad connecting to the at least one slipper, the cylindrical pad having greater width than the main body of the knee wrap.

15. A method for donning a motion restriction kit, comprising the steps of:
placing a first slipper having a plurality of laces over a first foot, the plurality of laces carried by a tab securable over the surface of the slipper;
tensioning the laces by pulling the tab; and
attaching the tab to a substrate to a surface proximate to the first slipper.

16. The method of claim 15, further comprising the step of placing a second slipper over a second foot.

17. The method of claim 16, further comprising the step of securing the tab of the first slipper to a surface of the second slipper, the surface of the second slipper forming the substrate.

18. The method of claim 16, further comprising the step of placing a cylindrical pad between the first and second slippers, a surface of the cylindrical pad forming the substrate upon which the tab secures.

19. The method of claim 15, further comprising the step of wrapping an ankle strap about a rear portion of the first slipper, the first slipper having an open rear end.

20. The method of claim 15, further comprising securing a knee wrap to a pair of legs, the knee wrap including a main body placed between the pair of legs, and a pair of loops each respectively secured about one of the legs.
